(19) United States
(12) Patent Application Publication
(10) Pub. No.: US 2025/0144319 A1
(43) Pub. Date: May 8, 2025

(54) ROTATOR FOR A MEDICAMENT DELIVERY DEVICE

(71) Applicant: SHL Medical AG, Zug (CH)

(72) Inventors: John Wiklund, Nacka Strand (SE); Andreas Lundqvist, Nacka Strand (SE); Robert Forsberg, Nacka Strand (SE); Per Tengliden, Nacka Strand (SE); Anders Boström, Nacka Strand (SE); Caroline Wannehed Bergström, Nacka Strand (SE); Martin Bue Bertelsen, Holte (DK); Gregor Manke, Unterhaching (DE); Allan Lester Kierkegaard, Kokkedal (DK)

(21) Appl. No.: 18/836,934

(22) PCT Filed: Jan. 26, 2023
(86) PCT No.: PCT/EP2023/051860

§ 371 Date: Aug. 8, 2024

Related U.S. Application Data
(60) Provisional application No. 63/405,521, filed on Sep. 12, 2022.
(60) Provisional application No. 63/308,122, filed on Feb. 9, 2022.

(51) Int. Cl. A61M 5/32; A61M 5/20

(52) U.S. Cl. CPC A61M 5/326; A61M 2005/2013; A61M 2005/208; A61M 2005/3267

(57) ABSTRACT

The present disclosure relates to a medicament delivery device comprising a housing having a proximal end and contains a pre-filled syringe positioned within the housing, where the syringe has a needle, a dose of medicament and a sliding stopper. A tubular activation member, e.g., a needle cover that is slidably positioned within a housing of the delivery device to move from a first position to a second position, and then to a final locked position. The needle cover has two distally projecting arms and a proximal end portion terminating in a radial flange. A plunger rod can only move proximally relative to the needle cover when the needle cover is in the second position. The radial flange is positioned outside of the proximal end of the housing when the needle cover is in the first position, the second position, and the locked position. The radial flange has an outer diameter D3 greater than the outer diameter of the proximal end portion D2, but less than the outer diameter D1 of the proximal end of the housing.

### TECHNICAL AREA

The present application relates to a medicament delivery device comprising an automatic dose delivery mechanism triggered by a delivery member guard when pressed against a dose delivery site.

### BACKGROUND

A large number of medicament delivery devices on the market and developed during the last 20 years are referred to as auto-injectors because they require pushing the device against a dose delivery or injection site to activate the device such that a dose of medicament can be administered or injected directly into a patient. Once activated a compressed drive spring is released that causes a plunger rod to expel the medicament out of the delivery member associated with a container of medicament. One such well known auto-injector is disclosed in U.S. Pat. No. 9,199,038. User comfort and ease of use of auto-injectors, as well as safety features, are areas where design changes are possible that improve the overall user experience and operational performance of such devices.

Specifically, the present disclosure is directed to improved structural components for contacting an injection site, improving the device activation experience, providing a smooth lockout feature to prevent user access to the used delivery member, and improving the robustness of the injection device as a whole.

### SUMMARY

In the present disclosure, when the term “distal” is used, this refers to the direction pointing away from the dose delivery site. When the term “distal part/end” is used, this refers to the part/end of the delivery device, or the parts/ends of the members thereof, which under use of the medicament delivery device is/are located furthest away from the dose delivery site, also referred to herein as an injection site. Correspondingly, when the term “proximal” is used, this refers to the direction pointing to the dose delivery site. When the term “proximal part/end” is used, this refers to the part/end of the delivery device, or the parts/ends of the members thereof, which under use of the medicament delivery device is/are located closest to the dose delivery site. Delivery member and injection needle are both used herein to refer to the component placed at the dose delivery site and that expels the medicament.

The aim of the present application is to remedy or improve upon perceived drawbacks of known auto-injection devices. This aim is solved by providing a more robust design that allows a user to experience a more responsive and smoother activation of the device. Additionally, the presently disclosed designs assist in manufacturing quality and results in an easier lockout feature that prevents accidental contact with the used delivery member or an attempted reuse of the device. Preferable embodiments of the present application form the subject of the dependent claims.

According to one aspect, the medicament delivery device of the present disclosure has a tubular housing that is open at both the proximal and distal ends. An actuator having an end cap is located at the distal end and is securely fitted into the distal end opening of the housing. A plunger rod is partially assembled and positioned within a tubular proximal portion of the actuator and initially is in a first biased position. The plunger rod is hollow and has a closed proximal end. A compressed drive spring is positioned within the plunger rod and biases the closed proximal end in a proximal direction. The tubular proximal portion of the actuator has a flexible finger that releasably locks the plunger rod when it is in the first biased position. A rotator is rotationally positioned around the tubular proximal portion of the actuator such that rotation of the rotator relative to the actuator, which is rotationally and axially fixed to the housing, causes the flexible finger to unlock from the plunger rod.

The rotator has an outer surface that is arranged with guide ledges, where some of the guide ledges are extending axially in the longitudinal direction (i.e., parallel to the longitudinal axis of the delivery device) and some are inclined in relation to the longitudinal direction. The guide ledges define one or more guide tracks that accepts and provides a pathway for one or more protrusions located on a distal end of a delivery member guard, also referred to herein as a needle cover. Axial travel of the needle cover and the protrusion within the guide tracks in a distal direction results in activation of the device. The guide track can have two or more non-linear paths that reduce the force needed to activate the device such that activation occurs when the rotator has completed rotation relative to the housing and actuator, and the guide track is again linear, i.e., parallel to the longitudinal axis of the delivery device. The completed rotation of the rotator causes release of the plunger rod from the locking engagement with the free ends of flexible arms arranged on the actuator. In one possible embodiment, rotation of the rotator occurs when the protrusion on the needle cover moving axially and distally in the guide track encounters a first inclined guide ledge. As the knob moves further distally, the protrusion passes the so-called point of no return and simultaneously engages a second inclined guide ledge. Having two or more inclined guide ledges reduces the activation force that is required and experienced by the user in order to initiation the automatic injection procedure.

The outer surface of the rotator can have a radially outwardly extending protrusion positioned on the end of a flexible finger that forms part of the locking mechanism that is activated once the delivery member guard is removed from the injection site after the dose of medicament is administered by the injection device. The other part of the locking mechanism is the protrusion on the distal end of the delivery member guard. As the delivery member is moved away from the injection site, the delivery member guard will be forced out of the proximal end of the housing by a biasing element, e.g., a compression spring. The protrusion will simultaneous move proximally within a guide track on the rotator until it rides up and over a wedge-shaped protrusion at the end of the flexible finger. The design of the flexible finger must allow enough flexibility to ensure that the protrusion will not bind and can easily move past the protrusion, i.e., slide up and over, so that the protrusion will be positioned behind, i.e., on the proximal side of the wedged-shaped protrusion. When in this position any attempted movement of the delivery member guard in the distal direction will be prohibited because the protrusion will impact and be stopped by the wedged shaped protrusion.

As mentioned, the medicament delivery device has a needle cover (delivery member guard) that is slidably positioned within the proximal end of the housing. The needle cover has a radial flange located at a terminal proximal end that is design for contact and engagement with an injection site, typically a preselected location of a user's skin tissue. The radial flange functions as a bearing surface when a user pushes the outer housing proximally towards the injection site. The outward extending radial flange also provides a greater contact surface area than earlier known needle cover designs. This increased contact (surface) area provides for a better user experience and comfort, especially for those users with more than average fatty tissue. This is especially true when compared to prior known needle covers that are devoid of any radial flange feature. In a preferred design, the outer diameter of the radial flange is greater than the outer diameter of the proximal tubular portion of the needle cover and less than the outer diameter of the housing and the interior diameter of the safety cap.

To allow for a maximum possible surface area of the proximal end face of the radial flange, while still allowing the use of a protective cap that attaches to the proximal end of the delivery device, the radial flange can have one or more notches around the periphery of the radial flange. The notches can be sized to accept longitudinal ribs positioned on an inside surface of the protective or safety cap. These notches can also be configured as improved attachment points to ensure that the safety cap remains removably attached to the delivery device before its intended use.

The delivery member guard can also have a distal end designed with two parallel longitudinally extending legs. The width and lengths of the legs are configured to maximize the robustness of the delivery member guard. Preferably, the thickness (T) of legs is increased about 19% over known similar longitudinal legs. Likewise, the delivery member guard of the present disclosure has increased widths (W2, W3) of about 8% and 7%, respectively. The distal ends of the legs are likewise configured with several features that ensure smooth functioning of the activation procedure when the delivery member guard is pushed against the injection site causing the delivery member guard to retract distally into the housing. These features can include rib guides that provide better slidability support because the rib guides are longitudinally positioned on the outside surface of each arm and form a channel configured to accept the ribs on the inside of the housing.

Another feature for enhance device operation includes housing ribs positioned longitudinally on the inside of the housing that project radially outward from the inside surface of the housing. These ribs have a projection thickness that cooperates with the rib guides on the distal end of the needle cover. The cooperation of the rib guides with the housing ribs prevents the delivery member guard from rotating relative to the housing and provides a guiding path or surface to ensure a smooth and non-binding sliding of the needle cover during activation of the device. Preferably the rib guides have a longitudinal length (L) that is approximately four times the length of similarly known rib guides. The length L is preferably proportioned to the stroke (travel) length of the needle guard during activation.

The protrusions mentioned above that are located on inner distal end surfaces of the longitudinally extending legs of the needle cover can be positioned and offset from the terminal end of the legs at a distance such that the activation of the device is either delayed or earlier as the radial flange is pressed against the injection site. Moving the protrusion in the distal direction will cause activation earlier. The protrusion can further include a chamfer or bevelled surface that is angled 20 degrees or less to prevent jamming/binding of the legs of the delivery member guard during activation as the protrusions slide in the track guide paths located on the outside surface of the rotator.

The present disclosure also can include a transport lock assembly for a powerpack of a medicament delivery device. In some circumstances the medicament delivery devices of the present disclosure can be delivered as multiple subassemblies that are connected together in a final assembly procedure. For example, in assembling an auto-injector with a loaded or compressed drive spring, one such subassembly may be a powerpack, where the compressed drive spring is biasing a plunger rod, where a rotator is configured to release the pre-tensioned plunger rod in the final assembled delivery device. For this reason, the rotator is movable, so that another element, typically part of a different sub-assembly, may interact with the rotator when the user of the medicament delivery device intends to administer a dose of medicament. During transport of the sub-assemblies, vibrations, movements and impacts may lead to accidental or unintentional release of the pre-tensioned plunger rods, which then renders the powerpacks useless for completing the device assembly. As such, it desired to ensure that the rotators are held securely or locked for shipment so that they do not accidentally activate the powerpacks during transport. At the same time, these locked powerpacks must not become difficult or complicated to unlock and/or to assemble as a result of transport locking security measures.

The present disclosure can include a transport lock assembly for the powerpack that is unlocked at the time of final assembly. One such transport lock assembly includes a powerpack having a spring-biased plunger rod, a body for holding the spring-biased plunger rod in a pre-tensioned state, a rotator for releasing the spring-biased drive member from the body, and a locking member configured to interact with the rotator, which locking member is movable, relative to the rotator, from a first state in which the rotator is immobilized, to a second state in which the rotator member is free to move, the transport lock assembly being further characterized by a housing part having a key member, which housing part is configured to receive the powerpack, and wherein assembly of the powerpack with the housing part causes the key member to move the locking member from the first state to the second state relative to the rotator.

Delaying the release of the rotator transport lock assembly is important in order to improve the injection and dose delivering functions. One design method to purposefully cause the delay is to provide a cut-out on the inside distal end of the housing of the delivery device. Such an improved design will prevent or delay rotation of the rotator during the assembly stroke so as to prevent the needle cover knob from being assembled into the wrong guide track, which in turn would make the injection device unusable. A shorter assembly stroke with an unlocked rotator would likely decreases the risk for wrong alignment.

To assist in the efficient assembly of the medicament delivery devices of the present disclosure several features can be included in the design, such as, the use of one or more visible markers than can be checked for alignment during the assembly process. For example, a marker can appear on the rotator and on an outer surface of the housing near an opening in the housing where the marker on the rotator can be viewed through the housing opening. Alignment of the two markers during the assembly process confirms the correctness of the assembly. The inclusion of chamfers and blocking gates can also assist in the assembly process. For example, including a combination of chamfers and blocking gates on the rotator outer surface can prevent misalignment of the legs of the delivery member guard when the power pack assembly 82, that includes the rotator, is inserted and fitted into the distal end of the housing.

A protective cap is releasably connected to the proximal end of the medicament delivery device and typically has a generally tubular body, a generally tubular medicament delivery member shield remover for removing the medicament delivery member shield. The medicament delivery member shield remover comprises an attachment element. The protective cap comprises a lid attached to the tubular body so that the medicament delivery member shield remover is held in place in this position by the end lid been in contact with the attachment element of the medicament delivery member shield remover. The attachment elements are for holding the generally tubular medicament delivery member shield remover. The lid is attached to said body for holding said medicament delivery member shield remover in a fixed position. This provides an easy and effective assembly setup of a safety cap.

Accordingly, in one possible embodiment of the present disclosure, a medicament delivery device has a housing having a proximal end and contains a pre-filled syringe positioned within the housing, where the syringe has a needle, a dose of medicament and a sliding stopper. A tubular activation member, i.e., a needle cover, is slidably positioned within the housing to move from a first position to a second position, and then to a final locked position. The needle cover has two distally projecting arms and a proximal end portion terminating in a radial flange. A resilient member, e.g. a compression spring, is located in the proximal end of the needle cover, where the spring is in a compressed state when the needle cover is in the second position. A plunger rod is operatively connected to the stopper and can only move proximally relative to the needle cover when the needle cover is in the second position. Each of the distally projecting arms comprises a distal portion that forms a lock when the needle cover is in the locked position such that the needle cover is prevented from moving proximally relative to the housing and exposing the needle. The radial flange is positioned outside of the proximal end of the housing when the needle cover is in the first position, the second position, and the locked position. The radial flange has an outer diameter D3 greater than the outer diameter of the proximal end portion D2, but less than the outer diameter D1 of the proximal end of the housing.

The radial flange can have two or more notches located around the outer circumference of the radial flange, where the notches are configured to accept longitudinal ribs on the inside surface of the safety cap.

Another aspect concerns a rotator for a medicament delivery device, the rotator comprising:

a tubular body extending from a proximal end to a distal end in an axial direction relative to a longitudinal axis; one or more ridges extending from a surface of the tubular body, the one or more ridges defining a track (211-213) on the surface of the tubular body, the track extending in the axial direction from a distal end of the track to a proximal end of the track, the track comprising one pathway at the distal end of the track and two pathways at the proximal end of the track, wherein the two pathways at the proximal end of the track are separated by at least one of the one or more ridges; and wherein a first pathway (211, 212) of the two pathways at the proximal end of the track is bounded at the distal end of the first pathway by a portion of the one or more ridges, wherein the portion is angled relative to the longitudinal axis, and wherein a first section (127a) of the portion is at a different angle relative to the longitudinal axis than a second section (127b) of the portion. Optionally, the first section of the portion is angled at a larger angle relative to the longitudinal axis than the second portion. Additionally or alternatively, the second section is closer to the distal end of the rotator than the first section. Additionally or alternatively, the first section is attached to the second section. Additionally or alternatively, the second section of the portion is angled at between 10 and 80 degrees relative to the longitudinal axis, or at between 20 and 70 degrees, or at between 25 and 50 degrees. Additionally or alternatively, the first section of the portion is angled at between 20 and 75 degrees relative to the longitudinal axis, or at between 30 and 70 degrees, or at between 30 and 60 degrees. Additionally or alternatively, the first section is angled at between 5 and 45 degrees more relative to the longitudinal axis than the second portion, or at between 5 and 35 degrees, or at between 10 and 30 degrees.

These and other aspects of, and advantages with, the present disclosure will become apparent from the following detailed description of the disclosure and from the accompanying drawings.

### BRIEF DESCRIPTION OF DRAWINGS

In the following detailed description of the disclosure, reference will be made to the accompanying drawings, of which

FIG. 1 is an exploded view of a medicament delivery device comprising a protective cap according to the application;

FIGS. 2 and 3 are cross-sectional views of the medicament delivery device of FIG. 1;

FIG. 4 is a detailed view of individual components that form the medicament delivery device of FIG. 1;

FIG. 5 is a detailed view of individual components that form the medicament delivery device of FIG. 1;

FIG. 6 is a detailed view of a syringe holder that forms part of the medicament delivery device of FIG. 1;

FIG. 7A is a detailed view of the needle cover of the medicament delivery device of FIG. 1;

FIG. 7B is a detailed view of individual components that form the medicament delivery device of FIG. 1;

FIG. 7C is a detailed view of the distal end of the needle guard of FIG. 7A;

FIG. 7D is a detailed view of individual components that form the medicament delivery device of FIG. 1;

FIG. 7E is a detailed view of individual components that form the medicament delivery device of FIG. 1;

FIG. 7F is a detailed view of individual components that form the medicament delivery device of FIG. 1;

FIG. 7G is a detailed view of individual components that form the medicament delivery device of FIG. 1;

FIG. 7H is a detailed view of individual components that form the medicament delivery device of FIG. 1;

FIG. 7I is a detailed view of individual components that form the medicament delivery device of FIG. 1;

FIG. 7J is a detailed view of individual components that form the medicament delivery device of FIG. 1;

FIG. 7K is a detailed view of individual components that form the medicament delivery device of FIG. 1;

FIG. 8 is a detailed view of individual components that form the medicament delivery device of FIG. 1;

FIG. 9A is a detailed view of the rotator of the device of FIG. 1;

FIG. 9B is a detailed view of guide tracks of the rotator of FIG. 9A;

FIG. 10 is a detailed view of individual components that form the medicament delivery device of FIG. 1;

FIG. 11 is a detailed view of individual components that form the medicament delivery device of FIG. 1;

FIG. 12 is a detailed view of individual components that form the medicament delivery device of FIG. 1;

FIG. 13 is a detailed view of individual components that form the medicament delivery device of FIG. 1; and

FIG. 14 is detailed view of the rotator unlocking sequence, with: the left portion showing part 1 of the rotator unlocking sequence, with the rotator lock still engaged (needle cover almost into rotator); the middle portion showing part 2 of the rotator unlocking sequence, with the rotator lock becoming disengaged; and the right portion showing part 3 of the rotator unlocking sequence, with the rotator assembled (disengaged).

### DETAILED DESCRIPTION

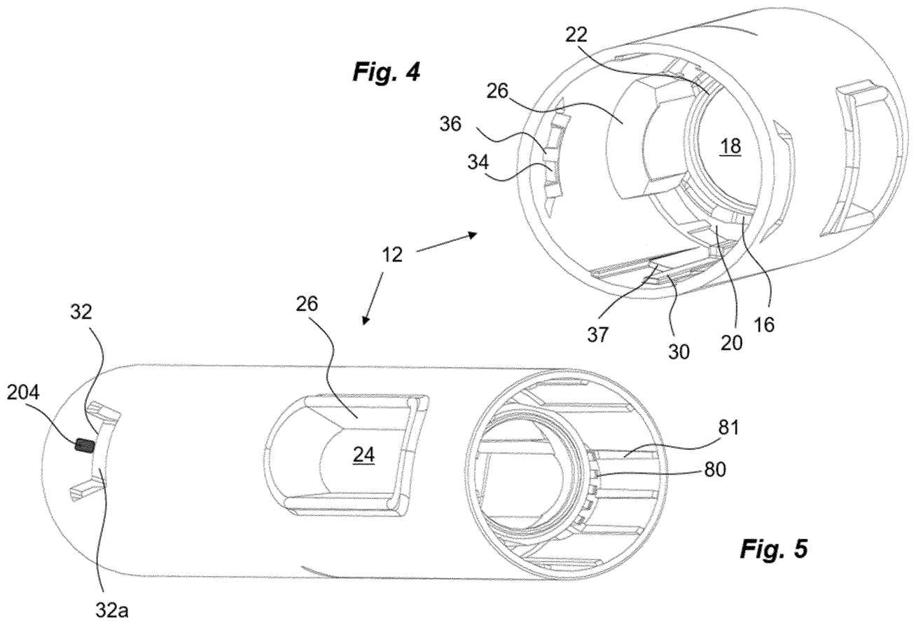

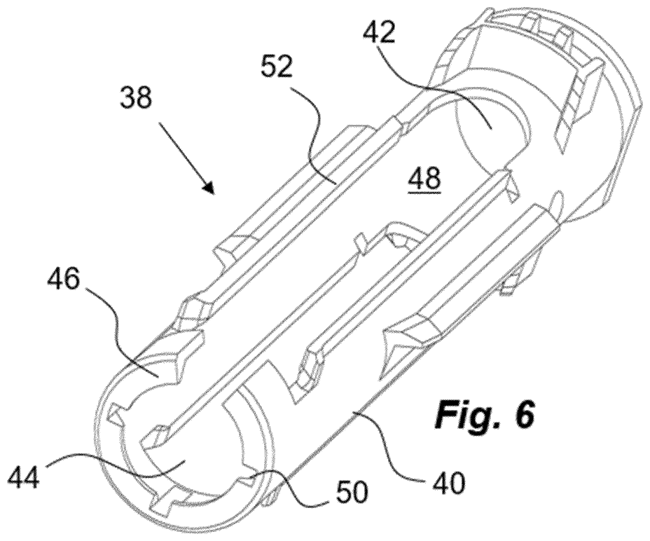

The medicament delivery device 10 shown in the drawings comprises a generally tubular housing 12 having a proximal end 14 and a distal end 15. Inside the housing a generally transversal wall 16 is arranged, FIG. 4, which wall 16 is provided with a central passage 18. Cut-outs 20 are further arranged on opposite sides of the passage 18. A seat 22 is surrounding the passage 18. Generally rectangular windows 24 are arranged in the housing, which windows 24 are arranged with inwardly directed wall sections 26. The proximal parts of the wall sections 26 are attached to or form part of the transversal wall 16. A number of longitudinally extending ribs 30 and 80 are arranged on the inner surface of the housing at both the proximal and distal ends of the housing. An inwardly directed protrusions 32 positioned as part of a cut-out at the distal end of the housing has generally radially flexing tongues 34 that are arranged with inwardly extending ledges 36 at their free ends. An assembly alignment indicator 204 is located on the outer surface of the proximal end of the housing 12 and is positioned near the protrusion 32 and associated cut-out portion of the housing

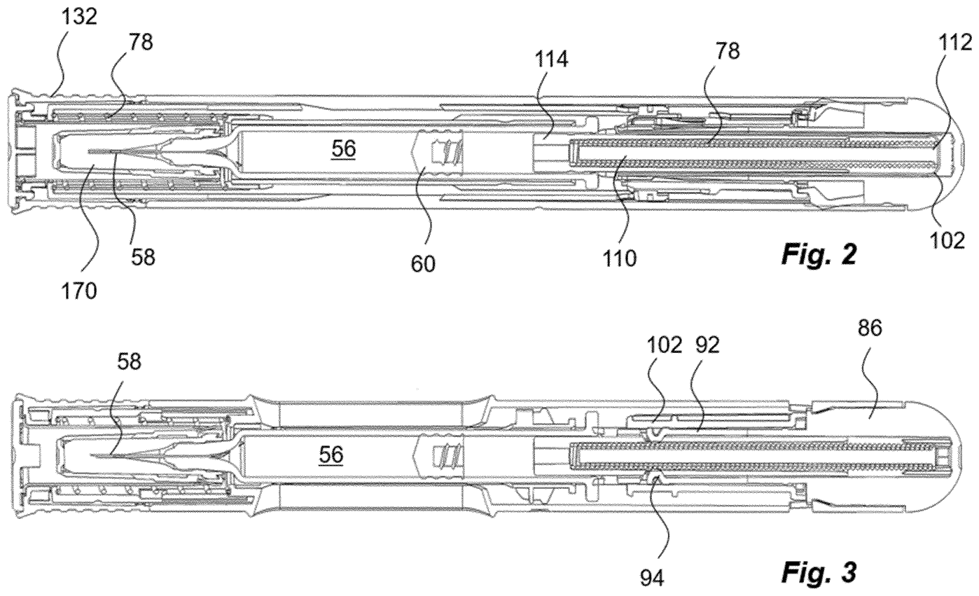

Inside the housing, a medicament container holder 38 is arranged coaxially. The medicament container holder 38 comprises a generally elongated tubular body 40 having a distal passage 42 and a proximal passage 44. The proximal passage 44 is arranged with an inwardly directed ledge 46 stretching around the circumference. The body 40 is arranged with two elongated slits 48 on opposite sides of the body. One of the slits 48 extends all the way to the proximal end, connecting the slit with the proximal passage, creating a C-shaped appearance when viewing in the distal direction. The circumferential ledge is further arranged with a number of cut-outs 50, three in the embodiment shown, for providing flexibility of the proximal part of the medicament container holder as will be described. The longitudinal sides of the slits 48 are arranged with outwardly directed ledges 52, which ledges 52 are designed to be in contact with inwardly surfaces 54 surrounding the windows 24 of the housing, for providing orientation and rotational fixation in relation to the housing. The medicament container holder 38 is arranged to accommodate a medicament container 56, which is shown in FIG. 2 as a pre-filled syringe, having an injection needle 58 as a medicament delivery member attached to a proximal end thereof and a stopper 60 of resilient material that is slidably movable inside the tubular body of the medicament container 56.

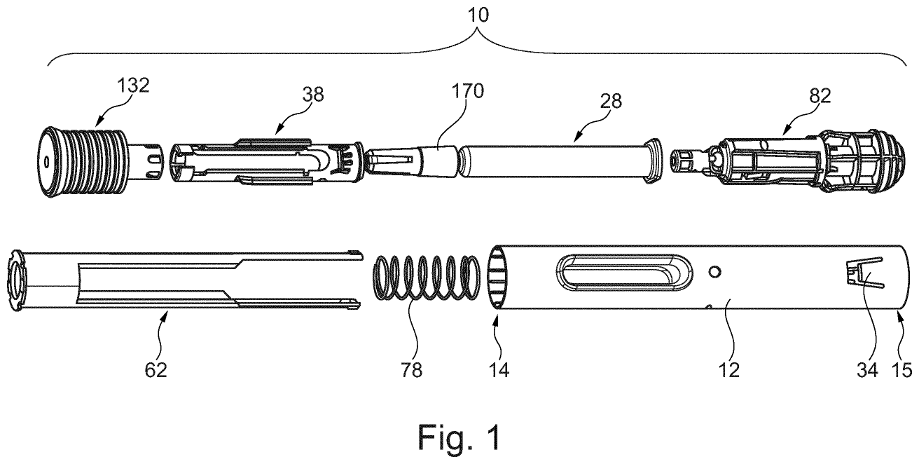

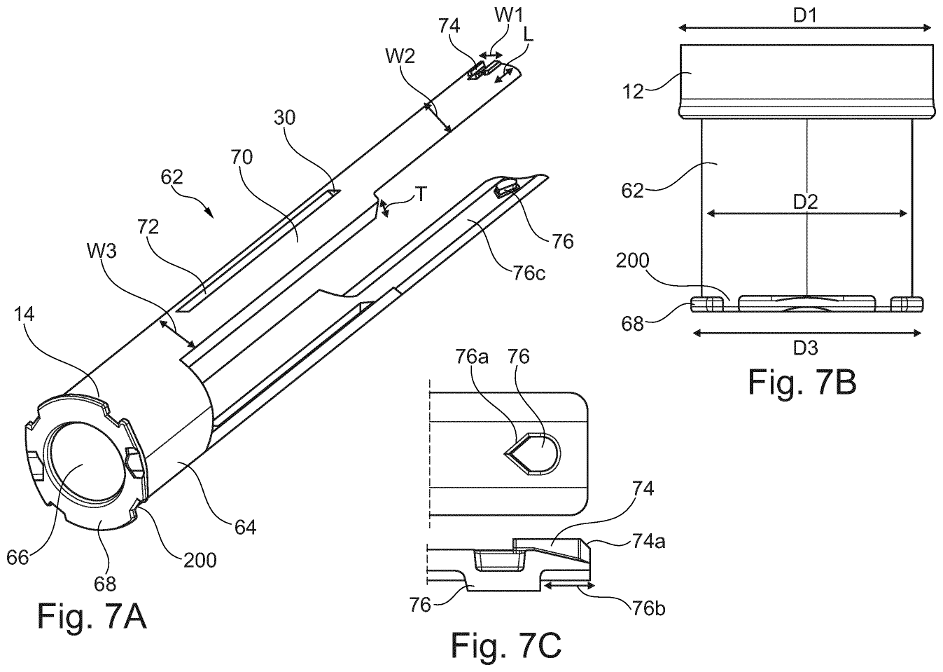

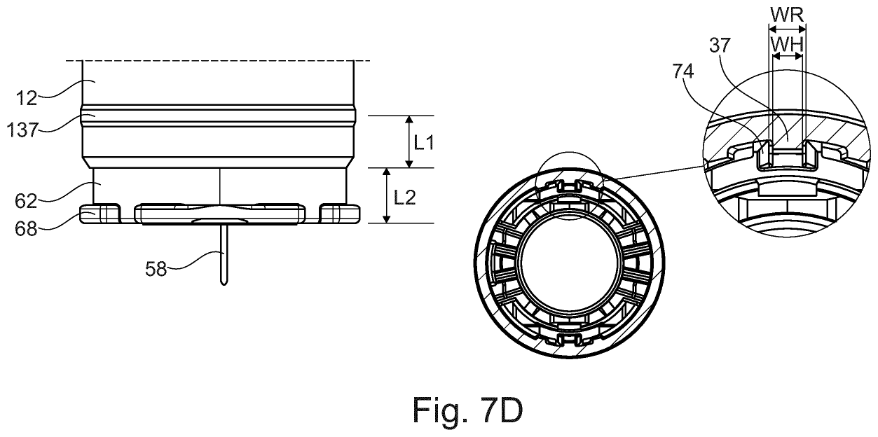

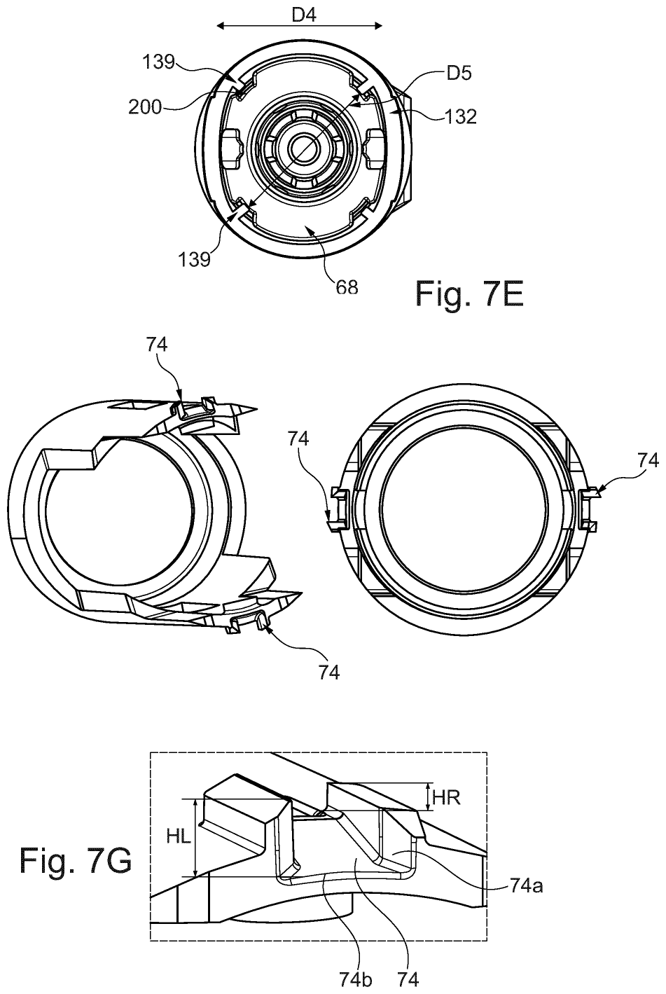

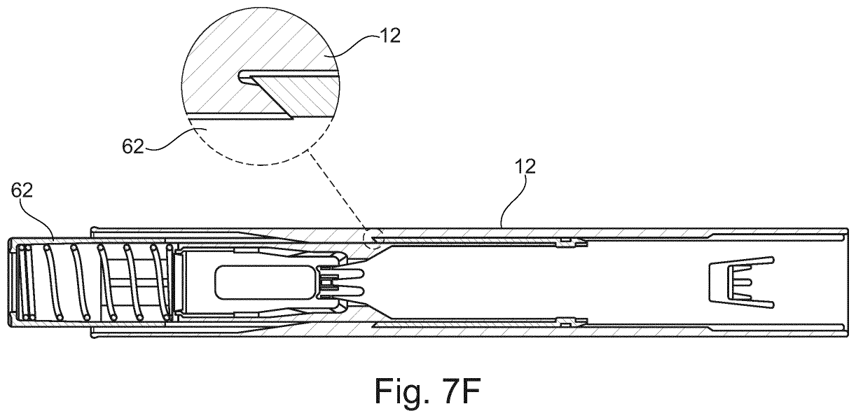

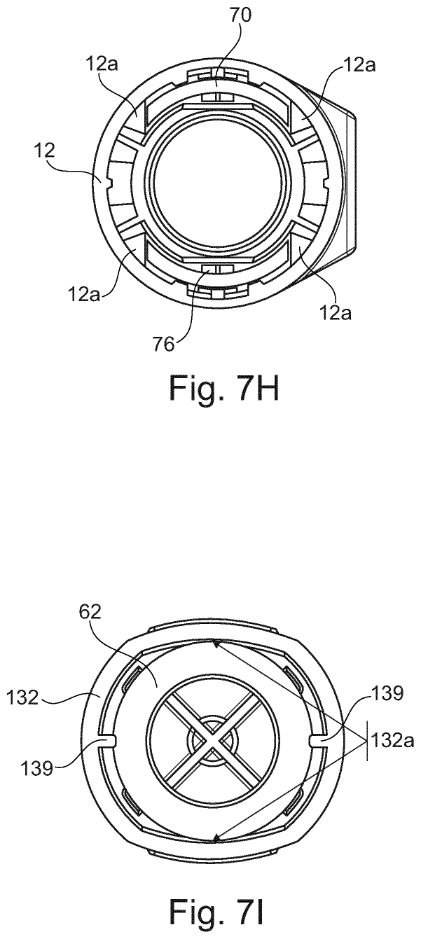

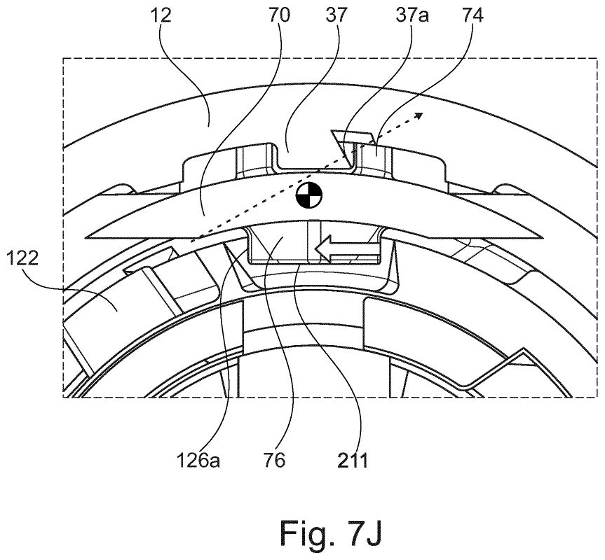

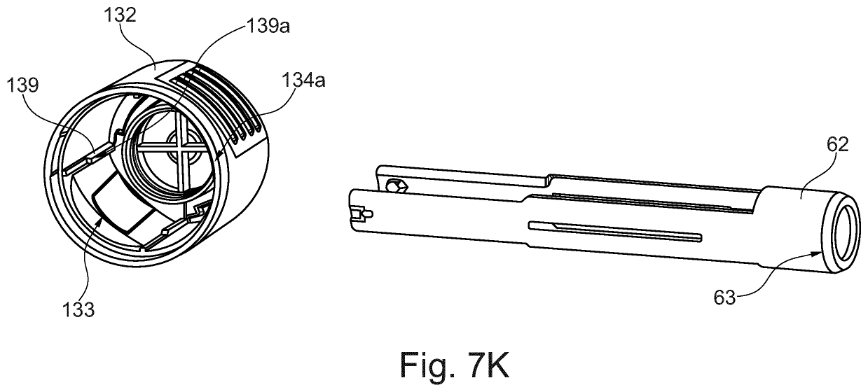

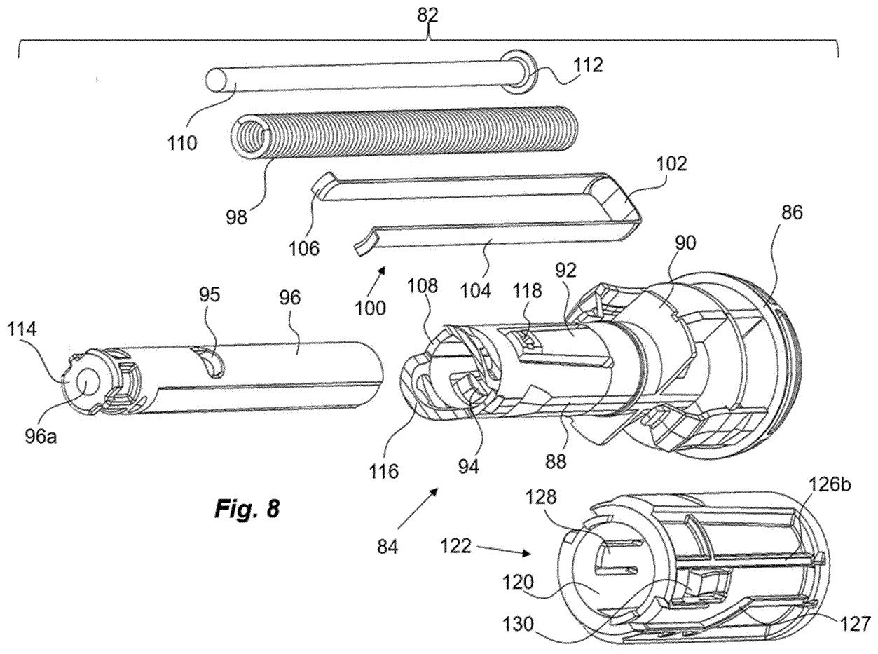

The medicament delivery device further comprises a medicament delivery member guard 62, also referred to as a needle cover (see FIGS. 1 & 7A). The delivery member guard 62 comprises a proximal generally tubular body 64 provided with a central passage 66 of a diameter to allow passage of needle 58 during medicament delivery. The needle guard 62 terminates at the proximal end in a transversal radial flange 68 that defines an injection site bearing surface. The radial flange 68 has an outside diameter D3 (see FIG. 7B) that is greater than the outside diameter D2 of the proximal end 64 but is less than the outer diameter of D1 of housing 12. Two or more notches 200 are located around the circumference of radial flange 68 and are sized to accept longitudinal ribs 139 located on the inside surface of safety cap 132 (see FIGS. 7E & 11). The ribs 139 define a diameter D5 that is less than the inside diameter D4 of cap 132, which is greater than the outside diameter D3 of radial flange 68. Engagement of ribs 139 and notches 200 prevents relative rotation of the cap 132 with respect to the housing 12 and needle cover 62. Cap 132 connects with housing 12 through rim 137 (see FIG. 7D).

An alternative safety cap 132 design is possible where two of the four ribs 139 are eliminated and the remaining two ribs are moved approximately 45 degrees from that shown in FIG. 7E. An example of such an alternate design is shown in FIG. 7I, where two ribs are placed 180 degrees opposite each other and the interior of cap 132 is flattened or made non-circular to create two contact points 132a, each located 90 degrees from ribs 139. These contact points 132a function as support walls for the proximal end of the needle cover 62.

Two oppositely positioned arms 70 are arranged to the distal area of the body 64 and extending in the distal direction. The arms 70 are arranged with longitudinal slits 72 which are to cooperate with the longitudinal ribs 30 of the interior of the housing (see FIG. 4). The arms 70 are characterized by two different widths, namely W2 and W3 (see FIG. 7A), where W2 is less than W3, and both widths are selected to provide sufficient rigidity to prevent binding or bending of the needle guard as the radial flange 68 is pushed against an injection site prior to completing activation of the medicament delivery device and commencing dose delivery. At the distal end of the arms 70, outwardly radial directed ledges 74 are provided. Preferably, two such ledges are used on each distal end of the arms 70 and are oriented and spaced longitudinally to define a channel having a width W1. This channel is sized accept longitudinal rib 37 on the inside of housing 12 (see FIG. 4) such that rib 37 functions as a guide track for the distal sliding movement of the needle guard relative to the housing as the delivery device is pressed and pushed against an injection site. As illustrated in FIG. 7D, in one possible embodiment, the ratio of the width of the radial directed ledges 74 (WR) to the width of the housing rib 37 (WH) is approximately 1.18.

Preferably, the tolerances between WR and WH are designed to be as small as possible to constrain radial movement of the needle guard relative to the housing 12. Employing narrow spatial tolerances between WR and WH greatly reduces the risk of derailing between the arms of the needle guard and housing during axial movement of the needle guard. Likewise, minimizing the ratio of WR:WH improves the gearing ratio between the needle guard and housing, which also greatly reduces needle guard arm twist and derailing. Another possible solution to prevent arm twist or derailing is illustrated in FIG. 7J which illustrates a geometric self-locking design, where housing rib 37 is configured with an angled engagement portion 37a that slidably engages an inside surface of ledge 72 on the needle guard. This angled engagement forces the radially inward directed protrusions 76 to slidably engage side walls 126,127 of guide track 211 thereby preventing needle guard arm twist and derailing.

In another possible design as illustrated in FIG. 7G, the height of the outwardly radial directed ledges 74 HL is maximized to better guide the axial movement of the needle guard as it moves distally along the longitudinal housing rib 37 (see FIG. 7J) which, although not shown, equals height HR as depicted, where the ratio of HL to HR is approximately 2.6:1, when HL is measured at the bottom of draft 74b. Ledge 74 also has guide wall interaction feature 74a that ensures the housing rib 37 smoothly enters the channel defined as the space between the ridges 74. This particular design also prevents flexing of the arms 70 of the needle guard as it moves axially in the distal direction. To further ensure non-binding or flexing of arms 70 as it moves axially relative to the inside of housing 12, preferably webs or walls 12a, as shown in FIG. 7H, are included on the inside surface of the house to minimize the space or create a tighter fit between the arms 70 and the housing.

On the inner surface of the arms 70 there are radially inward directed protrusions 76 (see FIG. 7C). The protrusions 76 are preferably tear dropped shape and has a bevelled or chamfered edge 76a less than 58 degrees, preferably 20 degrees or less, and most preferably 15 or 8 degrees, as measured relative to the inner surface 76c of the leg 70. These angled chamfers will prevent or minimize an undesirable failure mode of delayed activation. The distance of the protrusions is predetermined to achieve a desired needle injection depth. The protrusions 76 are positioned a distance 76b from the terminal distal end of legs 70. As indicated, the delivery member guard can also have a distal end designed with two parallel longitudinally extending legs. The width and lengths of the legs are configured to maximize the robustness of the delivery member guard. Preferably, the thickness (T) of legs is increased about 19% over known similar longitudinal legs (see FIG. 7A). Likewise, the delivery member guard of the present disclosure has increased widths (W2, W3) of about 8% and 7%, respectively. The protrusions 76 have been moved 0.25 mm to delay activation.

A medicament delivery member guard spring 78 is further arranged between a distally directed surface of the radial flange 68 of the needle cover 62 and a proximally directed surface of the wall 16. In this regard, proximally directed support protrusions 80 (see FIG. 5) are provided on the wall 16 for supporting the medicament delivery member guard spring 78 and preventing it from accidentally interacting with the arms 70 of the needle cover 62. The spring 78 biases the needle cover before, during and after activation of the delivery device and completion of the medicament medicant delivery at the injection site. FIG. 7D illustrates the spatial relationships of the housing 12, needle cover 62 and proximal end of the needle 58 when the radial flange 68 has been pressed against an injection site and medicament delivery has begun. The housing 12 and/or needle cover 62 lengths are preselected and designed such that L2 is 1.5 mm or greater so as to avoid contact of the terminal proximal end of the housing with the injection site, which can cause discomfort to the user. Likewise, rim 137 is located a distance L1 from the terminal proximal end of the housing to avoid interaction with the injection site. L1 is preferably 3 mm or greater.

The medicament delivery device 10 shown in FIG. 1 also comprises a power pack or drive unit 82. The power pack 82 comprises an actuator 84 (FIG. 8) provided with a distal portion forming an end cap 86 of the distal end of the housing 12 of the medicament delivery device when the power pack is inserted and fitted into the housing. The proximal part of the actuator 84 comprising a generally elongated tubular body 88. A transversal support surface 90 is arranged in the area between the end cap 86 and the body 88, which support surface 90 is designed to cooperate with the ledges 36 of the tongues 34 on the housing 12 for locking the actuator 84, and thus the power pack, to the housing 12 when the device is fully assembled. The body 88 is further arranged with proximally directed arms 92 that are flexible in a generally radial direction. The free ends of the arms 92 are provided with inwardly directed protrusions 94. These inwardly directed protrusions 94 are arranged to fit into and cooperate with recesses or cut-outs 95 in an elongated plunger rod 96, which plunger rod 96 is a hollow tubular structure having a closed proximal end and an open distal end, where the plunger rod is intended to fit into and be coaxial with the body 88 of the actuator 84.

The rotator 122 also can have an alignment indicator 206 that can be visible through cut-out 32a at the distal end of the housing 12. During device assembly of the power pack 82 into the housing 12, the rotator is positioned and oriented with respect to the housing such that alignment indicator 204 is aligned with alignment indicator 206 on the rotator. Alignment indicator 206 could be a small, but detectable, sink-notch, that is visible through cut-out 32a after final assembly and used to verify rotator 122 positioning throughout the life cycle of the device.

Further, a drive spring 98 is arranged partially inside the hollow portion 96a of the plunger rod 96 as well as a guide rod 110 is arranged inside the drive spring 98, where the guide rod is provided with a disk 112 at its distal end. A U-shaped bracket 100 having a transversal distal part 102 and two proximally extending arms 104 on either side of, and outside, the drive spring 98. The ends of the arms 104 are arranged with outwardly extending ledges 106, which ledges 106 are to be in contact with proximally directed edge surfaces 108 of the body 88 of the actuator 84. The drive spring 98 is thus arranged between a proximal end wall 114 of the plunger rod 96 and the transversal distal part 102 of the bracket 100 via the disk 112 of the guide rod 110 (See FIGS. 2 & 8). Further, at the proximal end of the body 88 there are arc-shaped support elements 116 that project proximally and are flexible in the generally longitudinal direction. These elements 116 are intended to be in contact with and support the medicament container 56 so as to minimize relative axial movement of the container 56 relative to the medicament container holder 38. Preferably, the elements 116 exert a biasing force in the proximal direction to prevent unwanted axil movement of the container 56. In other words, the contact or engagement by elements 116 can impart a biasing force in the proximal direction on the medicament container holder 38 and/or the medicament container itself.

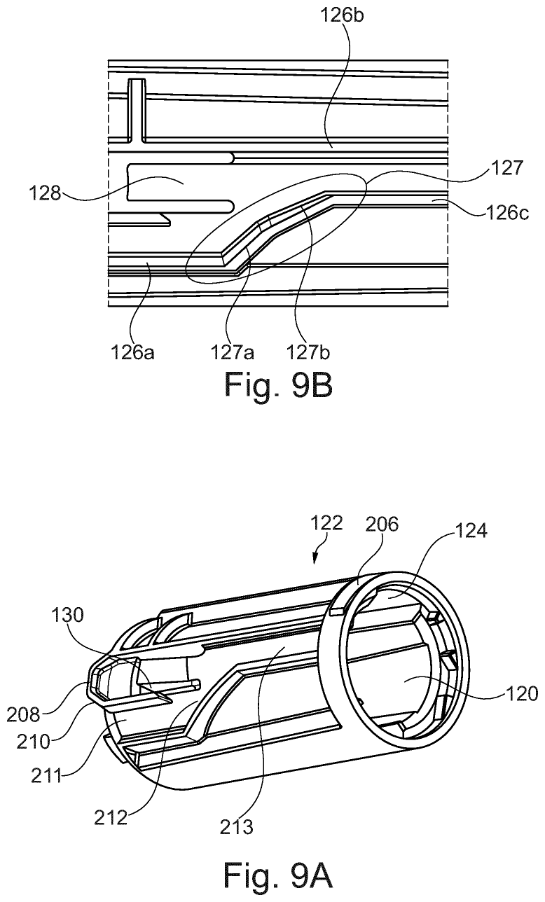

The free ends of the arms 92 of the body 88 are arranged with outwardly directed protrusions 118 that are intended to cooperate with inner surfaces 120 of a generally tubular rotator 122 that is arranged outside and coaxial with the body 88 of the actuator 84. The inner surface 120 of the rotator 122 is arranged with longitudinally extending grooves 124, FIG. 9A, the function of which will be described below. The outer surface of the rotator 122 is arranged with a plurality of guide ledges or ribs 126a-c and 127a-b that define one or more guide tracks 211-213, where some of the guide ledges are extending in the longitudinal direction 126a, 126b, & 126c and some are inclined as represented by section 127 in relation to the longitudinal direction as will be explained. Adjacent one longitudinal guide rib 126b, there is a proximally directed tongue 128 is arranged, which tongue 128 is flexible in the generally radial direction, and where the free end of the tongue 128 is arranged with an outwardly directed, wedge-shaped, protrusion 130.

To activate the medicament device of the present disclosure the needle cover 62 must be pressed against an injection site causing it to retract into the housing 12 where the protrusions 76 will move towards the proximal end of rotator 122 such that the protrusion 76 will enter track 211 (see FIGS. 9A-B). To prevent misalignment or jamming of the sliding motion of the needle cover 62 relative to the housing 12, the edge 208 and angle edge 210 are provided to guide the protrusion 76 into track 211. The needle cover will move first along the track 211 following guide ledge 126a. Initially, the user must only overcome the biasing force of compression spring 78. However, as the needle cover is pushed further into the housing the protrusion 76 will encounter the angled change in the wall of the guide ledge 127a followed by a second angled change caused by guide ledge 127b, both defining guide track 212, prior to entering guide track 213. As the needle cover is pushed along the angled changes the spring 78 is further compressed. This requires the user to exert more force than would be needed than simply further compressing the spring 24 along a straight linear track. Engagement with the angled guide ledges causes the rotator 122 to start to rotate clockwise relative to the needle cover, the body 88 and the housing 12. As the protrusion travels past the first angled guide ledge 127a the user will notice a drop in the required exertion force as the needle cover moves past the point of no return. Further axial movement in the distal direction of needle cover along the second angled guide ledge 127b causes further rotation of the rotator 122. Once the rotator has completed rotation and the protrusion 76 will then align and enter guide track 213. At this point, the exertion force drops off further and then continues to increase as the compression of spring 24 is further compressed until the needle cover achieves the retracted position as indicated in FIG. 7D.

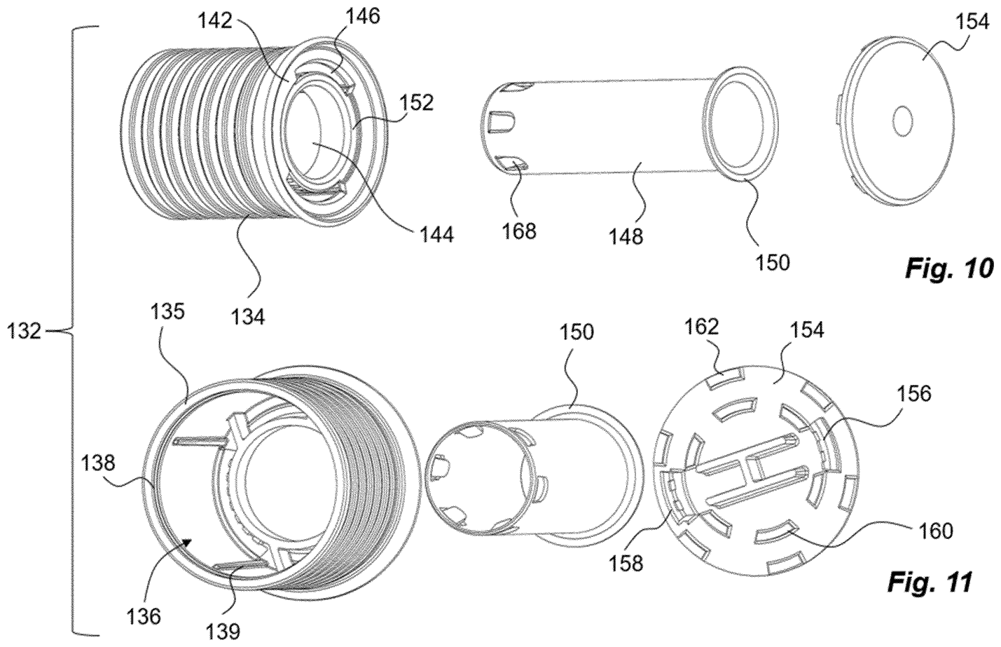

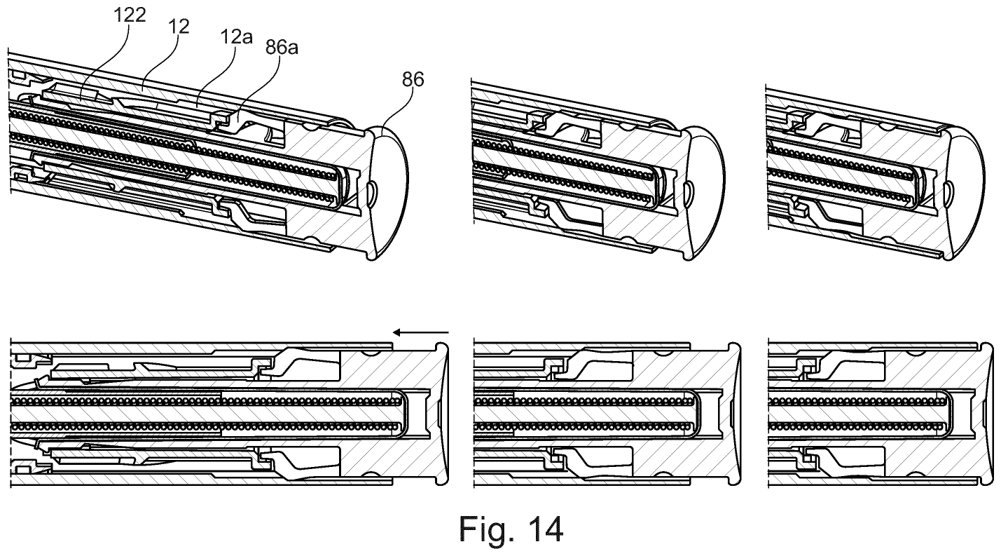

The medicament delivery device 10 is further arranged with a safety cap 132, FIGS. 1 and 10-11, comprising a generally tubular body 134 having a distal passage 136. In order to provide a good fit between the safety cap 132 and the housing 12 of the medicament delivery device 10, the inner surface of the body 134 of the safety cap 132 may be arranged with a circumferential ledge 138, which ledge 138 is arranged to interact with protrusions 140, FIG. 7, on the outer surface of the body 64 of the medicament delivery member guard 62 as seen in FIG. 14. The body 134 of the safety cap 132 is further arranged with a distally directed end surface 135 (see FIG. 11) that acts as an abutment surface against a proximally directed end surface, e.g., rim 137 (FIG. 7D) of the housing 12, which rim 137 also acts as an abutment surface such that the surfaces 135, 137 provide a specific position of the protection safety cap 132 when mounted onto the medicament delivery device 10.

The body 134 of the safety cap 132 is arranged with a proximal end wall 142, which end wall 142 is arranged with a central circular passage 144. Radially outside the central passage 144 are two oppositely positioned arc-shaped openings 146. Alternatively, only one opening of a suitable shape can be provided (not shown here). A generally tubular medicament delivery member shield remover 148 is to be positioned in the central passage 144 of the end wall 142, wherein the medicament delivery member shield remover 148 will extend into the body 134 of the safety cap 132. The proximal end of the medicament delivery member shield remover 148 is arranged with an outwardly extending ledge 150, which ledge or flange 150 is arranged to be seated in a seat or a recess 152 in the end wall 142 of the body 134. The medicament delivery member shield remover 148 is held fixed in place in this position by an end lid 154. The end lid 154 is arranged with a couple of distally directed arc-shaped arms 156 as illustrated in FIG. 11, provided with radially outwardly directed ledges 158, wherein the arms 156 are designed to fit into the arc-shaped openings 146 of the body 134 and the ledges 158 will snap around edges of the arc-shaped openings 146, locking the end lid 154 to the body 134 of the safety cap 132. Alternatively, only one distally directed arm of a suitable shape or an arm comprising a couple of flexible fingers (now shown) can be arranged. The end lid 154 is further arranged with one or a number of distally directed protrusions or ledges 160 which are to be in contact with the ledge 150 of the medicament delivery member shield remover 148, holding it in place in the recess 152.

The distal end of the medicament delivery member shield remover 148 is arranged with generally proximally and inwardly inclined tongues 168 that are designed to be in contact with and engage a medicament delivery member shield 170 such as a rigid needle shield or a flexible needle shield, covering the medicament delivery member such as the injection needle 58. The protective safety cap has a facilitated assembly procedure in that the medicament delivery member shield remover 148 is simply entered into the central passage 144 of the end wall 142 of the body 134 from the proximal side until the ledge 150 is seated in the recess 152 in the end wall 142. Then the end lid 154 is simply pushed onto the proximal end of the body 134 such that the arms 156 fit into the openings and the ledges 158 snap around the edges of the openings 146 of the body 134. A very simple, fast and effective assembly way of the protective safety cap is obtained by the solution.

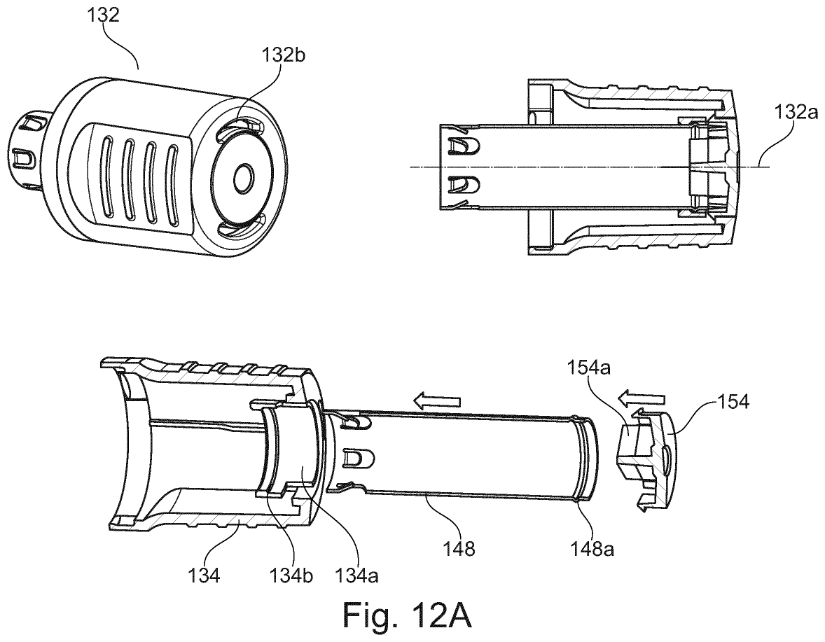

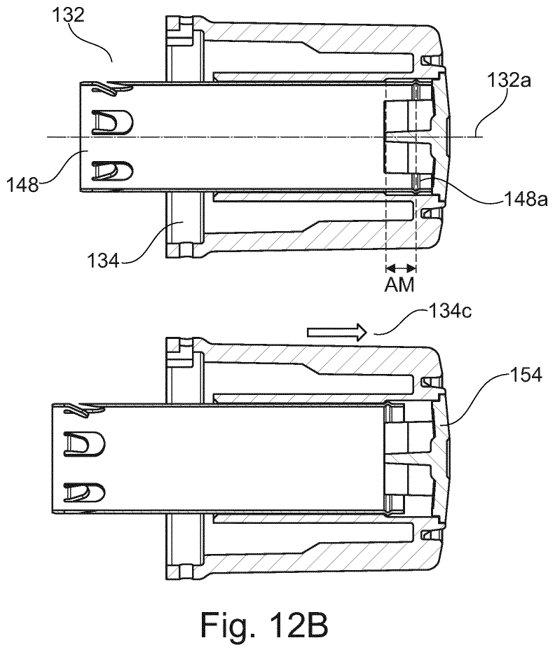

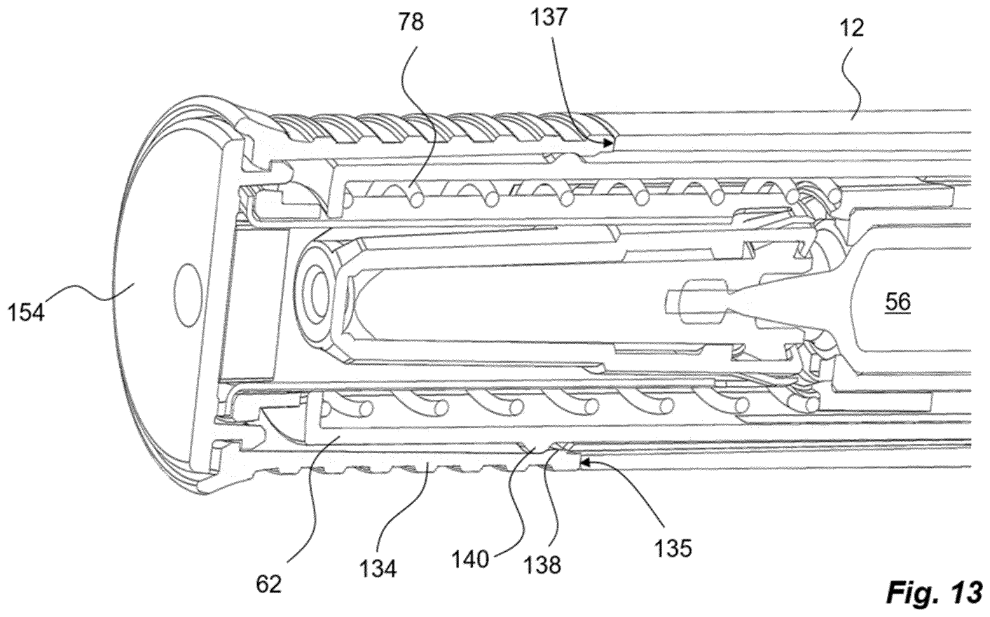

Yet another possible design of the safety cap assembly 132 is illustrated in FIGS. 12A and 12B. Although this design is also as a three-component configuration, this design has a distally facing circular shield remover connector 134a that has an extended groove 134b on an inner portion that is configured to allow for easy engagement with a corresponding outer protrusion 148a on the proximal end of the delivery member shield remover 148. As indicated by the directional arrows in FIG. 12A, the shield remover is attached to the body 134 first followed by insertion and connection of the end lid 154. This simplifies the assembly of safety cap assembly 132, yet ensures that the shield remover is securely axially fix to body 134 when the user removes the safety cap assembly from the housing of the delivery device.

The outer protrusion 148a is sized to form a secure snap fit with inner extended groove 134b to axially fix the remover shield relative to the inside of the proximal end of body 134 when the safety cap is being removed from the housing of the delivery device. As illustrated in FIG. 12B, there are two axial positions of the body 134 relative to the remover 148. The first is the assembled position where the remover has been inserted through the distal end of the body 134 and the end lid 154 has been inserted into the through hole at the distal end of body 134 to hold the remover in place. In this assembled position the distal end of the remover is bottomed out on the end lid to define an axial movement gap AM between the outer protrusion 148a and the inner extended groove 134b. This gap AM allows for axial movement of the body 134 relative to the remover as the user begins the process of removing the safety cap assembly from the delivery device housing. When the body has moved to the second axial position relative to the remover the inner extended groove 134b will then engage with the outer protrusion 148a such that continued axial movement of the body will also cause axial movement of the remover. Having the gap AM makes it easier, i.e., requiring less force, for a user to remove the safety cap assembly from the housing of the delivery device because initially there is only movement of body without any movement the remover and RNS off of the syringe. The size of gap AM is predetermined such that the body releases from the housing of the delivery device before the remover begins to move axially with the body 134.

With reference to FIG. 12B, the RNS remover stands on the bottom of the cap at syringe assembly. During removal of the cap the RNS, the RNS remover will move to a second position and allow the cap to be released before the RNS starts to move relative to the syringe.

The design of the outer protrusion 148a and the inner extended groove 134b is preferably one that allows rotation of the safety cap body 134 relative to remover 148 but prevents relative axial movement once the protrusion 148a and groove 134b are engaged when the body is in the second axial position. The distally extending ribs 154a on the inside surface of end lid 154 prevent or greatly reduce canting, tilting or otherwise moving out of alignment of the shield remover with and relative to the longitudinal axis 132a of the cap assembly 132. The cap assembly shown in FIG. 12A also has openings 132b on the proximal end of the body 134 that provides an air passage through the safety cap 132 and prevents possible suffocation should a child for example put the safety cap 132 in the mouth.

FIG. 14 illustrates one possible embodiment of a rotator transport locking feature 86a that can be used during device assembly to prevent premature or unwanted rotator rotation, which as explained above can cause misalignment of the distal ends of the needle guard with the guide tracks of the rotator. Preferably, the inside of the housing 12 comprises deep grooves 12a at the distal end that becomes shallower as the grooves extend towards the proximal end of the housing. The varying depth of the groove 12a allows the power pack assembly 82 of the injector, which includes the rotator component 122, to be fed further (e.g., 3-4 times longer distance than previously possible) into the housing until the rotation lock 86a on rear cap 86 disengages and the rotator 122 can rotate freely. The needle cover protrusion is then very close to the axial position of the guide track opening on the rotator 122 when the rotation lock 86a is disengaged. The rotator unlocking sequence is shown in FIG. 14.

The medicament delivery device according to the drawings is intended to function as follows. The medicament delivery device is delivered to a user with the safety cap 132 attached to the proximal end of the medicament delivery device. The medicament delivery member guard 62 is in an extended position in relation to the housing 12 such that when the abutment surface 135 of the safety cap 132 is in contact with the abutment surface 137 of the housing, the circumferential ledge 138 is distally of, and in contact with, the protrusions 140 of the medicament delivery member guard 62 as seen in FIG. 14. This provides a very secure fit, reducing the risk for premature release of the safety cap 132.

In order to ensure smooth, non-binding assembly of the safety cap 132 onto the housing 12 of the delivery device, it is desirable to reduce or eliminate non-essential physical contact between the needle guard 62 and the inside of safety cap 132. This can be accomplished by including a chamfered edge 63 at the of the proximal end of needle guard 62 (see FIG. 7K). Likewise, the inside surface 133 of cap 132 can be configured not to have excess wall material that can rub, bind or otherwise engage with the proximal end of the needle guard. Similarly, the inwardly projected surfaces 139a of the longitudinal ribs 139 can be configured so that there is minimal or no engagement with the proximal end of the needle guard. Increasing the contact surface area 134a of distal end of the safety cap 132 is preferred to ensure a secure fitting with the housing 12.

The medicament delivery device is generally activated by the needle cover 62 being pushed into the housing 12 when the radial flange 68 is pressed against a dose delivery, e.g., injection, site, as will be described. In certain circumstances, activation can be unintended. For example, this may happen accidentally if the medicament delivery device is dropped against a hard surface such as a floor. Such an accident could cause a risk that the medicament delivery device 10 is activated in that the needle cover 62 may be moved in relation to the housing 12 due to the impact forces, which might trigger the medicament delivery device. This risk is reduced or eliminated in that the needle 62 is held by the engagement with the safety cap 132 by the protrusions 140 interacting with the ledge 138.

Once the safety cap is removed, along with the needle shield, the user can then press the radial flange 68 against the injection site, whereby the needle cover 62 is pushed into the housing 12, causing a penetration by the injection needle 58. The movement of the needle cover 62 will cause the protrusions 76 at the distal end of the needle cover to slide in relation to the rotator 122. As the protrusions move along the guide tracks on the outside surface of the rotator, the rotator will rotate in relation to the actuator 84, which in turn causes the outwardly protrusions 118 of the arms 92 of the actuator 84 to be moved in position with the longitudinal grooves 124 on the inner surface 120 of the rotator 122. The arms 92 are thereby free to move radially outwards, whereby the engagement between the inwardly directed protrusions 94 and the recesses 95 of the plunger rod 96 is removed, releasing the plunger rod 96. The plunger rod 96 is then urged in the proximal direction by the force of the compressed drive spring 98. The plunger rod 96 will now act on and move the stopper 60 of the medicament container 56 in the proximal direction, expelling a dose of medicament through the injection needle 58. At the end of the injection sequence, the distal end of the plunger rod 96 will pass the bracket 100 whereby the arms 104 of the bracket 100 are free to move radially inwards, wherein the ledges 106 are moved out of contact with the surfaces 108 of the actuator 84. Because the distal end of the drive spring 98 is in contact with the transversal distal part 102 of the bracket 100 via the disk 112 of the guide rod 110 and since the drive spring 98 has a residual force, the bracket 100 will be forced suddenly in the distal direction until the distal end of the bracket 100 hits an end wall of the actuator 84, causing a tactile and audible signal to the user that the injection sequence is completed and that it is safe to remove the medicament delivery device from the dose delivery site.

The user can now remove the radial flange from the injection site which then allows the needle cover 62 to be pushed in the proximal direction by the medicament delivery guard spring 78 that was initially compressed as a result of the needle cover being push in the distal direction during contact of the radial flange and the injection site. The biasing force in the proximal direction caused by the spring 78 decompressing will cause the needle cover and protrusions 76 to move proximally in the guide track 213 such that they come in contact with and pass the wedge-shaped protrusions 130 of the tongues 128 of the rotator 122. The protrusions 76 will ride up and over protrusion 103 until it stops on the proximal side of tongue 128. Passing of the protrusions 130 by protrusions 76 will result in the tongue 128 acting as a hard stop and preventing the protrusions 76 from moving axially in the distal direction. Thus, the needle cover 62 becomes locked in the extended position, covering the injection needle 58, in turn preventing accidental injuries on the injection needle 58. The medicament delivery device can now be discarded.

It is to be understood that the embodiment described above and shown in the drawings is to be regarded only as a non-limiting example that may be modified in many ways within the scope of the patent claims.

1-9. (canceled)

10. A rotator for a medicament delivery device, the rotator comprising:
a tubular body extending from a proximal end to a distal end in an axial direction relative to a longitudinal axis; and
one or more ridges extending from a surface of the tubular body, the one or more ridges defining a track on the surface of the tubular body, the track extending in the axial direction from a distal end of the track to a proximal end of the track, the track comprising one pathway at the distal end of the track and two pathways at the proximal end of the track, wherein the two pathways at the proximal end of the track are separated by at least one of the one or more ridges,
wherein a first pathway of the two pathways at the proximal end of the track is bounded at the distal end of the first pathway by a portion of the one or more ridges, wherein the portion is angled relative to the longitudinal axis, and wherein a first section of the portion is at a different angle relative to the longitudinal axis than a second section of the portion.

11. The rotator of claim 10, wherein the first section of the portion is angled at a larger angle relative to the longitudinal axis than the second portion.

12. The rotator of claim 10, wherein the second section is closer to the distal end of the rotator than the first section.

13. The rotator of claim 10, wherein the first section is attached to the second section.

14. The rotator of claim 10, wherein the second section of the portion is angled at between 10 and 80 degrees relative to the longitudinal axis.

15. The rotator of claim 10, wherein the second section of the portion is angled at between 20 and 70 degrees relative to the longitudinal axis.

16. The rotator of claim 10, wherein the second section of the portion is angled at between 25 and 50 degrees relative to the longitudinal axis.

17. The rotator of claim 10, wherein the first section of the portion is angled at between 20 and 75 degrees relative to the longitudinal axis.

18. The rotator of claim 10, wherein the first section of the portion is angled at between 30 and 70 degrees relative to the longitudinal axis.

19. The rotator of claim 10, wherein the first section of the portion is angled at between 30 and 60 degrees relative to the longitudinal axis.

20. The rotator of claim 10, wherein the first section is angled at between 5 and 45 degrees more relative to the longitudinal axis than the second portion.

21. The rotator of claim 10, wherein the first section is angled at between 5 and 35 degrees more relative to the longitudinal axis than the second portion.

22. The rotator of claim 10, wherein the first section is angled at between 10 and 30 degrees more relative to the longitudinal axis than the second portion.

23. A medicament delivery device comprising the rotator of claim 10.

24. The medicament delivery device of claim 23, wherein the medicament delivery device is an autoinjector.
